(19) United States
(12) Patent Application Publication
(10) Pub. No.: US 2025/0364124 A1
(43) Pub. Date: Nov. 27, 2025

(54) DECISION SUPPORT TOOL FOR HEALTH PRACTICES

(71) Applicant: TRUE CHARGE, LLC, Sarasota, FL (US)

(72) Inventors: Jacob S. LEWIS, Amherst, NY (US); Brett LINDENBERG, Satellite Beach, FL (US)

(21) Appl. No.: 19/219,079

(22) Filed: May 27, 2025

Related U.S. Application Data
(60) Provisional application No. 63/651,665, filed on May 24, 2024.

(51) Int. Cl. G16H 40/20; G16H 10/60; G16H 50/30; G16H 50/70; G16H 70/60

(52) U.S. Cl. CPC G16H 40/20; G16H 10/60; G16H 50/30; G16H 50/70; G16H 70/60

(57) ABSTRACT

A decision support system for healthcare workflow management including a computer system having a controller, a memory, and a user-interface. The system has a patient database wherein patients are uniquely identifiable and wherein records include at least sought procedures and sought date of appointments for those procedures. The system further has a medical professional database wherein medical professionals are uniquely identifiable and where records include at least skillsets and a calendar. At least one software program is designed to, substantially in real time, assess the calendar and the sought procedures, select changes in the calendar to schedule an appointment set for the sought procedures to the medical professionals and is further designed to weigh performance variables to maximize key performance indicators within resource constraints wherein expected value and variance at a beginning of a period are benchmarked against actual value and variance at period end.

### CROSS-REFERENCE TO RELATED APPLICATIONS

This application claims priority to and the benefit of U.S. provisional application 63/651,665, titled DECISION SUPPORT TOOL FOR HEALTH PRACTICES, filed on May 24, 2024, which is incorporated herein by reference in its entirety.

### FIELD

The present invention generally relates to a decision support system and method for real-time healthcare workflow management.

### BACKGROUND

There are various Electronic Health Record/Electronic Medical Record (“EHR” or “EMR”) vendors and scheduling software Vendors that provide basic components that cover logistics of surgical scheduling, but these solutions lack elements that are intended to optimize a surgeon's or organization's cash flows as effectively as they could. These solutions only cover basic elements of surgical scheduling such as location, date, time, procedure duration, equipment, instrumentation, operating room space, documentation of medical clearances and patient consent, and documentation of prior authorization. They did not solve the problem of cash flow variance in surgical practices.

Medical practices are often beset by large fluctuations in monthly collected revenue, despite the owner physicians performing consistent surgical volume year after year. Variances for a single practitioner can be several hundred thousand dollars per month, even though the total number of procedures performed year after year are about the same. The decline in revenue was multifactorial. For illustration, high paying insurance carriers in states for workers compensation and no-fault insurance policies for motor vehicle accidents can be slow to pay claims at that point in time, and the physician reimbursement fee schedules of health insurance, workers compensation, and no-fault carriers vary from one plan to the next. Each individual procedure, or part of a procedure, can be worth a variable amount, depending on how much each insurance plan pays for it. Often, it is challenging for physician practices to be financially sustainable with only Medicare Advantage and Traditional Medicare reimbursement. Thus, it is important for physician practices to have a portion of their patient population on commercial plans, especially if the practice is not accepting workers compensation or no-fault. However, practices may lack commercial patients as they tend to be younger and healthier than the Medicare and Medicaid patient populations. Further, manual approaches are too slow to be effective given the many random variables that must be considered and calculated, leaving much of scheduling based on intuition and, therefore, the best guess. Existing EHR systems lack computational algorithms to dynamically optimize schedules based on real-time reimbursement and resource data, leading to inefficient processing and unreliable outcomes.

Therefore, there is a need in the market for an improved scheduling tool for health practice logistics designed to balance patient sources and minimize cashflow variances in real time.

### SUMMARY OF THE INVENTION

Disclosed is a decision support system for healthcare workflow management including a computer system having a controller, a memory, and a user interface. The system has a patient database wherein patients are uniquely identifiable and wherein records include at least sought procedures and sought date of appointments for those procedures. The system further has a medical professional database wherein medical professionals are uniquely identifiable and where records include at least skillsets and a calendar. At least one software program is designed to, substantially in real time, assess the calendar and the sought procedures, select changes in the calendar to schedule an appointment set for the sought procedures to the medical professionals. The at least one software program is designed substantially in real time to weigh performance variables to maximize key performance indicators within resource constraints wherein expected value and variance at a beginning of the at least one period are benchmarked against actual value and actual variance at an end of the at least one period.

In one embodiment of the decision support system for healthcare workflow management has the patient database designed to store uniquely identifiable patient records wherein each record includes at least sought medical procedures and requested appointment dates. The medical professional database is designed to store uniquely identifiable professional records, including at least skillsets and calendars of available time slots. The at least one software program adapted to, substantially in real time, assess the calendar and the sought procedures, select changes in the calendar to schedule an appointment set for the sought procedures to be performed by given medical professionals, wherein the at least one software program is executed by the controller and configured to:
a) process, in real time, the calendar data from the medical professional database and procedure data from the patient database to generate an optimized schedule for the sought medical procedures, b) calculate, based on historical reimbursement data stored in the memory, an expected financial value and variance for a scheduling period, including such points as value and timeliness,
c) adjust the schedule by evaluating performance variables, including procedure-specific reimbursement rates and resource availability, to minimize the financial variance while maximizing at least one key performance indicator, including care timeliness, within resource constraints,
d) benchmark the expected financial value and variance against actual financial value and variance at the end of the scheduling period, and
c) store the benchmarked results in the memory to refine future schedule optimizations.

The software program is designed to reduce computational overhead by streamlining real-time data processing, thereby improving the reliability of healthcare workflow management. At least one performance variable other than earnings may be maximized by the software program for said scheduling period.

In some embodiments, at least one performance variable other than earnings is designed to be maximized at least to a threshold of performance by the software program per the at least one period. Upon at least reaching that threshold of performance, the software program is designed to maximize earnings. In some embodiments of the decision support system the value maximized is timeliness of care. In some embodiments of the decision support system the value maximized is a care outcome score.

In some embodiments of the decision support system at least one software program is a machine learning program trained to predict optimal schedules based on historical reimbursement data. In some embodiments of the decision support system, at least one software program is designed to receive patient care authorizations. In some embodiments of the decision support system, there is at least one surgical scheduler spreadsheet produced as output.

These and other objects, features, and advantages of the present invention will become readily apparent upon a review of the following detailed description of the invention, in view of the drawings and appended claims.

### BRIEF DESCRIPTION OF THE DRAWINGS

The nature and mode of the operation of the present invention will now be more fully described in the following detailed description of the invention taken with the accompanying drawing figures. It should be understood that these drawings only depict preferred embodiments of the present invention and are not therefore to be considered limiting in scope, thus, the invention will be described with additional specificity and detail through the use of the accompanying drawings in which:

FIG. 1 illustrates a representative decision support system;

FIG. 2 illustrates a representative decision cycle;

FIG. 3 illustrates a representative decision tree;

FIG. 4 illustrates a representative report;

FIG. 5 illustrates a representative reporting process;

FIGS. 6A and 6B illustrate a representative decision support method; and

FIGS. 7A-7D illustrate a second representative decision support method.

### DETAILED DESCRIPTION OF THE INVENTION

The following are detailed descriptions of various related concepts related to, and embodiments of, methods and apparatus according to the present disclosure. It should, however, be understood that this disclosure is not limited to the particular methodology, materials, and modifications described and, as such, may, of course, vary. It is also understood that the terminology used herein is for the purpose of describing particular aspects only and is not intended to limit the scope of the claims.

Furthermore, it should be appreciated that drawings are representative to illustrate the inventive concepts herein and may not be to scale. Also, like drawing numbers on different drawing views identify identical, or functionally similar, structural elements where there could appear some variations on exactness where exactness is not material to the inventive concept herein. For illustration, the type of head on a helically threaded connector could differ on like identified items when the importance is that the identified item is a helically threaded connector, and other items could be treated similarly. It is to be understood that the claims are not limited to the disclosed aspects.

Unless defined otherwise, all technical and scientific terms used herein have the same meaning as commonly understood to one of ordinary skill in the art to which this disclosure pertains. It should be understood that any methods, devices, or materials similar or equivalent to those described herein can be used in the practice or testing of the example embodiments.

It should be appreciated that the term “substantially” is synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. It should be appreciated that the term “proximate” is synonymous with terms such as “nearby,” “close,” “adjacent,” “neighboring,” “immediate,” “adjoining,” etc., and such terms may be used interchangeably as appearing in the specification and claims. It should be appreciated that the term “distal” and comparably related terms denoting further-away portions of an item are antonymous to proximal portions of the co-described item as those portions of items may be termed. The term “approximately” is intended to mean values within ten percent of the specified value.

It should be understood that the use of “or” in the present application is with respect to a “non-exclusive” arrangement unless stated otherwise. For example, when saying that “item x is A or B,” it is understood that this can mean one of the following: (1) item x is only one or the other of A and B; (2) item x is both A and B. Alternately stated, the word “or” is not used to define an “exclusive or” arrangement. For example, an “exclusive or” arrangement for the statement “item x is A or B” would require that x can be only one of A and B. Furthermore, as used herein, when referring to a set or group of items, for illustration (A, B, C) the term “at least one or more . . . and . . . ” such as in “at least one or more of A, B, and C” is intended to include any to all of the denoted set or group of items, i.e. it could include just one item from the set or group, it could include all of the items from the set or group, and it could include any other combination of the set or group of items that is greater than one item and less than all of the items, the illustrated example having three items meaning there are up to seven non-ordered combinations A, B, C, AB, AC, BC, ABC. Other numbers of items would have maximum combination possibilities calculated accordingly.

Moreover, as used herein, the phrases “comprises at least one of” and “comprising at least one of” in combination with a system or element is intended to mean that the system or element includes one or more of the elements listed after the phrase. For example, a device comprising at least one of: a first element; a second element; and, a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element. A similar interpretation is intended when the phrase “used in at least one of:” is used herein.

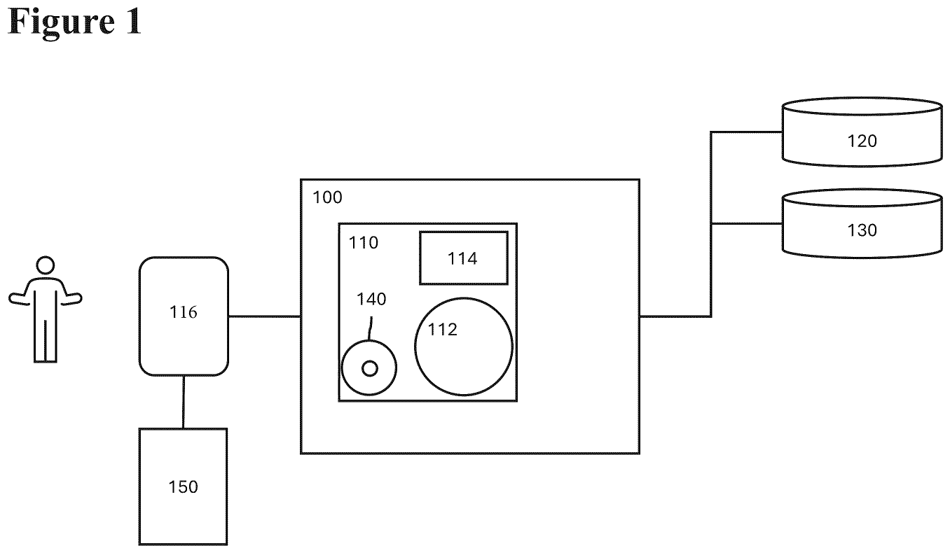

Disclosed in FIG. 1 is a decision support system 100 for healthcare workflow management including a computer system 110 having a controller 112, a memory 114, and a user interface 116. This computer system 110 could be a dedicated computer or could be a portion of a computerized device such as a handheld computer. The system has a patient database 120 wherein patients are uniquely identifiable and wherein records include at least sought procedures and sought date of appointments for those procedures. The system further has a database of one or more medical professionals 130 wherein medical professionals are uniquely identifiable and where records include at least skillsets and a calendar. Both databases could be collocated with the computer system 110 or could be accessed remotely, and the databases may be distributed or centralized.

At least one software program 140 is configured to execute a real-time optimization algorithm that processes calendar data from medical professional database 130 and procedure data from patient database 120 to dynamically generate an optimized schedule. Software program 140 is designed to evaluate performance variables, including procedure-specific reimbursement rates, resource availability (e.g., operating room capacity), and patient appointment preferences, to assign procedures to medical professionals within resource constraints. Software program 140 is designed to calculate an expected financial value and variance for a scheduling period based on historical reimbursement data and iteratively adjusts the schedule to minimize variance while maximizing key performance indicators, such as care timeliness and financial stability. At the end of the period, decision support system 100 benchmarks the expected value and variance against actual outcomes, storing the results in memory 114 from which to refine future optimizations. This operation of decision support system 100 is designed to reduce computational overhead of traditional scheduling systems by streamlining data processing, improving predictions, and improving, therefore, schedule reliability.

Decision support system 100 implements a software-based optimization algorithm as a part of software program 140 that processes multiple random variables to generate a schedule minimizing financial variance in healthcare practices. Random variables, such as X=x, meaning random variable X could take any of the possible values that x can take at a probability ranging from 0 and 1, represent specific data points (e.g., reimbursement amounts for a procedure), where x takes values within a probability range from 0 to 1 based on historical reimbursement data stored in memory 114. Conditional probabilities, such as P (X=x|Y=y), are computed, where Y, for illustration, could represent a surgical procedure (e.g., one of a predefined set of procedures in database 120) and x could represent reimbursement amounts contingent on an insurance carrier. Software program 140 evaluates probabilities in real time to assign procedures to medical professionals' calendars, optimizing predictable revenue by balancing high- and low-reimbursement procedures. This computational approach is designed to ensure low variance in cash flow, enabling reliable coverage of monthly operating expenses while adhering to resource constraints, such as operating room availability. By processing independent variables (procedures and insurance carriers) in a structured database framework, the system reduces the computational complexity of traditional scheduling, improving real-time performance, wherein machine learning may further refine algorithms associated with software program 140. The invention is designed to schedule specific types of procedures and types of health insurance plans in a manner that would produce predictable and reliable collected revenue where variance is low enough to ensure paying for monthly operating expenses based on commonly performed surgical procedures and the corresponding reimbursement for each of those procedures and according to several different health insurance plans and fee schedules.

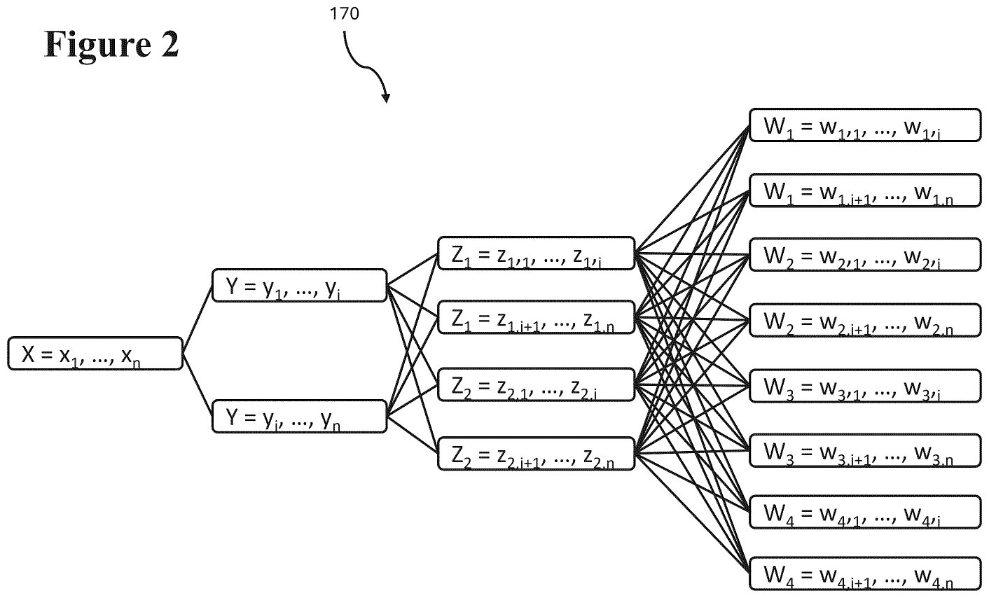

FIG. 2 illustrates a representative decision tree where, for illustration, random variable X might be a set of all common procedures x where X=x. The representative decision tree processes random variables stored in databases 120 and 130, where, for example, random variable X represents a set of common procedures (x₁, x₂, . . . , xₙ), and X=x denotes a specific procedure. At the next decision tree node, random variable Y represents a discrete set of insurance carriers (y₁, y₂, . . . , yₘ), where Y=y corresponds to a carrier with a reimbursement probability P (Y=y| X=x) derived from historical data in memory 114 and where y may itself have a range of probable values depending on the carrier and the procedure x. A third variable might be random variable Z where z might be a discrete set of patients needing procedure X, and W might be a fourth random variable where w might be a range when patient z is willing and able to receive procedure x. The variable i in this example represents a division of a set where members of the set less than or equal to i have one characteristic and members of the set greater than i have another characteristic. For illustration, one division of a set might, as a characteristic, be insurance plans that pay out above or below a given rate for a procedure or one division might be patients who have been waiting longer or shorter than a time threshold for receiving treatment. The outputs may be viewable in a spreadsheet 150 where expectation E[X]=Σₓx·p(x) is conditioned upon many preceding random variables, as illustrated, conditioned on preceding variables (e.g., P (X=x|Y=y, Z=z, W=w)). Other billing modifiers may be inserted, as well as split fee arrangements, and non-routine elements may be inserted into a set. For example, a non-routine procedure may be an xₙ₊₁ member of a set of procedures X=(x₁, x₂, . . . , xₙ)·p(x) for a given calculation. This is to say that the decision tree used for a given calculation may be generated from discrete sets of elements, elements that themselves may have probability ranges, and non-routine elements may be included in a calculation if needed where, with the addition, the sum of probabilities still equals 1. Further, calculations are typically, but not limited to, daily, weekly, monthly, and annually, where as time periods grow longer, non-routine procedures may become a distinct value x from all possible values of x with a historical range of values.

In some embodiments, at least one performance variable other than earnings is designed to be maximized by the software program 140 per the at least one period. In some embodiments, at least one performance variable other than earnings is designed to be maximized at least to a threshold of performance by the software program 140 per the at least one period. Upon at least reaching that threshold of performance, the software program 140 is designed to maximize earnings. In some embodiments of the decision support system 100 the value maximized is care timeliness.

For further illustration, some uses of the disclosed invention may be critical care or an emergency room where timeliness is of particular importance. In some embodiments of the decision support system 100 the value maximized is a care outcome score. For example, many institutions, though as a business they may wish to maximize revenue, may view that strategically, maximizing outcomes by an outcome score is the right thing to do and may strategically also be the superior way to maximize earnings over the long term. These embodiments illustrate that the decision support system 100 is designed to handle nuance where though the overall direction is to maximize earnings and reduce variance to that cash flow is smoothed during that period. The decision support system 100 can prioritize on other values first where it is designed to maximize earnings within the limitations set forth by prioritized values such as the above timeliness and outcome score.

In some embodiments, software program 140 includes a machine learning model trained to predict optimal scheduling configurations by analyzing historical scheduling data, reimbursement outcomes, and patient care metrics stored in databases 120 and 130. Said learning model refines its predictions over time by incorporating feedback from actual financial and care outcomes, improving accuracy of variance minimization. Software program 140 is further configured to interface with external systems to receive real-time patient care authorizations, integrating this data into the optimization process to ensure compliance with healthcare regulations. Additionally, decision support system 100 generates surgical scheduler spreadsheet 150 as output, formatted for human readability and compatible with existing healthcare management software, enhancing interoperability and usability.

The disclosed invention necessarily involves random variables where a variable, such as the earnings for a one-hour appointment, may vary depending on how that hour is filled. The expected value of a random variable X is a measure of the center of its distribution or mean. It represents the mean value that the variable is expected to take over many repetitions of given time cycles where each time cycle has a similar period. A time cycle, for example, might be a given day or week. Mathematically, if X is a random variable with probability distribution p(x), then the expected value of X, denoted as E[X] or μ, is calculated as the sum of each possible value of X weighted by its probability: E[X]=Σₓx·p(x) In the representative embodiment, random variables may be defined such as the types of procedures a medical professional might perform or the rate that might be charged for a given procedure or subset of procedures, the rate which may include hourly, by procedure, or other methods of determining how much to charge. Where variables are less distinct and exhibit continuous properties, integral variations of the above calculations may be used. Where multiple random variables may be involved, for instance, a probability that a given patient might be able to accept a scheduling change Y before determining X the X|Y may present the conditional probabilities that could occur, which may further be multiple conditional probabilities such as YN Z where Y is the availability of the patient and Z is the availability on schedules of a suitable medical professional for the procedure and X|(Y∩(Z), but such conditional variables—and possible independent variable—still, ultimately, undergo the base consideration that there is an expected value E[X] and which will have some degree of variance, that expected value can be expended to incorporate other variable—i.e., in the above example it could be represented as E[X|(Y∩Z)], and where G might be a representative or substitute variable for combinations such as X|(Y ∩Z) that there will be an actual value g from all possible values of g, Σ_{g}p(g), where G=g and some degree of variance, wherein the system is designed to maximize earnings within constraints governed by the legal, ethical, and enterprise obligations to deliver sought and timely healthcare to patients while also guarding against variance non-conducive to ongoing operations. Further, by pre-computing probability distributions and storing results in memory 114, decision support system 100 reduces the computational burden of iterative scheduling, enhancing the efficiency of real-time workflow management.

Variance in disclosed embodiments measures the spread or dispersion of the random variable's distribution and can account for illustration, that there might be a targeted value for delivering one or more medical procedures that may differ from the actual value obtained. Variance may happen for many reasons, such as scheduling flexibilities, availability of tools, whether a procedure was harder or easier than normal, what rates can be charged to given insurance carriers, who is conducting the procedure, and more. Such reasons may have an inherent randomness to them, but some probabilities, such as that a procedure will be covered by one insurance carrier versus another, may be higher than others and may be predicted based on past experiences or on information about the present and likely future states elements impacting probabilities. Variance quantifies how much the values of a random variable differ from the expected value. Mathematically, if X is a random variable with expected value E[X], then the variance of X, denoted as Var[X] or σ2, is calculated as the average of the squared differences between each value of X and the expected value: Var[X]=E[(X−E[X])²]. This can also be written as: Var[X]=E[X²]−(E[X])² where E[X²] is the expected value of X², calculations encoded in software program 140, designed process data such as procedure-specific reimbursement rates and resource availability from databases 120 and 130, and such equations will be represented in software code used. Other ways to illustrate the underlying mathematics may be presented, including calculations and presentations of standard deviations, but the operations will be similar to these representations. Calculations of variance may involve many variables, as illustrated by X|(Y∩Z), where G may be a representative or substitute variable wherein the variance for G=g is determined. This approach further optimizes computational resources by leveraging pre-stored probability distributions, enabling faster processing of complex, multi-variable scheduling tasks compared to traditional systems.

The variance component of the invention is designed to foster predictability and stability in managing cash flows wherein higher paying procedures, which often pay more slowly, and lower paying procedures, which often pay more rapidly are evenly distributed throughout the schedule and some priority can be given both to patients on higher and sometimes slower paying health insurance plans as well as patients on lower and sometimes faster paying health insurance plans where available without compromising timeliness of care for patients regardless of their source of financing, it generally being in the interest of all parties to have financially solvent medical professionals and so that the given user is not waiting on a few high-paying cases to cover its routine operating expenses. For example, using reimbursement rate data, decision support system 100 may compute Var[X|Y], where Y denotes insurance carrier type, to prioritize schedules that reduce financial fluctuations while ensuring compliance with care timeliness constraints stored in memory 114. By structuring a surgical schedule with even distributions of higher paying clients where available, and, therefore, limiting variance, users have an improved opportunity to treat every patient fairly by providing comparable lead times for surgery dates.

These underlying concepts are encoded into software programs and the mathematical representations disclosed herein are representative, and other methods of calculations and other terms may be used such as noted standard deviations. Other methods to obtain actionable numbers may be used, to include simple observation, graph plotting, and regression analyses but where again, an expected value, and actual value, and a variance will be determined.

Where values such as timeliness and outcome score take priority over earnings in some fashion, conditions may be included. For illustration, if earnings is the variable X and Y is a variable associated with timeliness than X|Y may be conditional that for all possible values of y, Σ_{y}, where Y=y that y<t where t is a threshold of time a patient has been waiting. Otherwise, where at least one value y>t, then Y=y even though X is not maximized. Such calculations are designed to take place in the background for the given user who, from the user interface 116, may be able to input, for illustration, to prioritize anyone who has been waiting for a given procedure for some period even if in that instance potential earnings are not maximized—and where, the system may provide recommendations where the user approves or disapproves those recommendations.

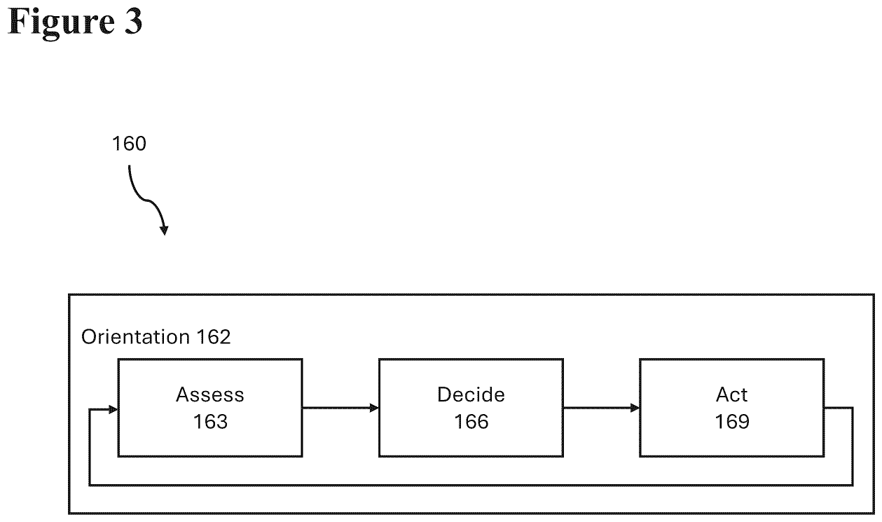

FIG. 3 illustrates a decision cycle 160 from which analyses are framed in the invention as a decision support tool operable substantially in real time. The decision cycle includes an assessment phase 163 wherein expected values are set. Decision 166 is a phase wherein procedures are scheduled. Act 169 is a phase where the procedures are conducted wherein a new assessment phase 163 begins including comparing expected values and variances with actual values and variances. The system is designed, therefore, to proficiently execute decision cycles 160 iteratively as fast as possible to a threshold of relevance definable as a time period (t) wherein an added orientation 162 portion concerns what values will be maximized and in what priority. One object of the invention as a decision support system is to improve decision cycles 160 whereby users maximize earnings and minimize variances within constraints that also best serve patients receiving treatment.

Decision cycle 160 leverages illustrated probability calculations implemented by software program 140 to process random variables and optimize healthcare scheduling in real time, as illustrated in FIG. 3. During assessment phase 163, software program 140 computes expected financial values, E[X]=Σxx·P(x), for a scheduling period, where X represents reimbursement amounts derived from procedure and insurance carrier data stored in databases 120 and 130. Conditional probabilities, such as P(X=x|Y=y), where Y, for example, denotes a specific procedure or insurance carrier, are evaluated to set expected values that account for resource constraints, such as operating room availability. Software program 140 also calculates expected variances, Var[X]=E[(X−E[X])²], to quantify potential financial fluctuations, enabling decision support system 100 to prioritize schedules that minimize cash flow variance while ensuring timely patient care. These computations, executed by controller 112, streamline the assessment phase by pre-processing historical reimbursement data stored in memory 114, reducing computational latency and enhancing the efficiency of real-time decision-making by way of improved decision cycles 160.

In decision phase 166, software program 140 applies computed probabilities and variance metrics to dynamically adjust medical professionals' calendars, assigning procedures to optimize key performance indicators, such as care timeliness and financial stability, as defined in orientation phase 162. For example, software program 140 balances higher-paying, slower-reimbursing procedures with lower-paying, faster-reimbursing procedures by evaluating conditional variances, such as Var[X|Y∩Z], where Y is patient availability and Z is medical professional availability. During act phase 169, procedures are performed, and actual financial outcomes are recorded. Subsequent assessment phase 163 compares actual values and variances against expected values set in prior cycles 160, storing results in memory 114 to refine future probability distributions, data which may further be used for training machine learning. This iterative process, driven by the decision cycle 160, integrates probability mathematics designed, therefore, to improve the reliability of healthcare workflow management, ensuring equitable patient treatment and stable cash flows within the constraints of legal and ethical healthcare obligations.

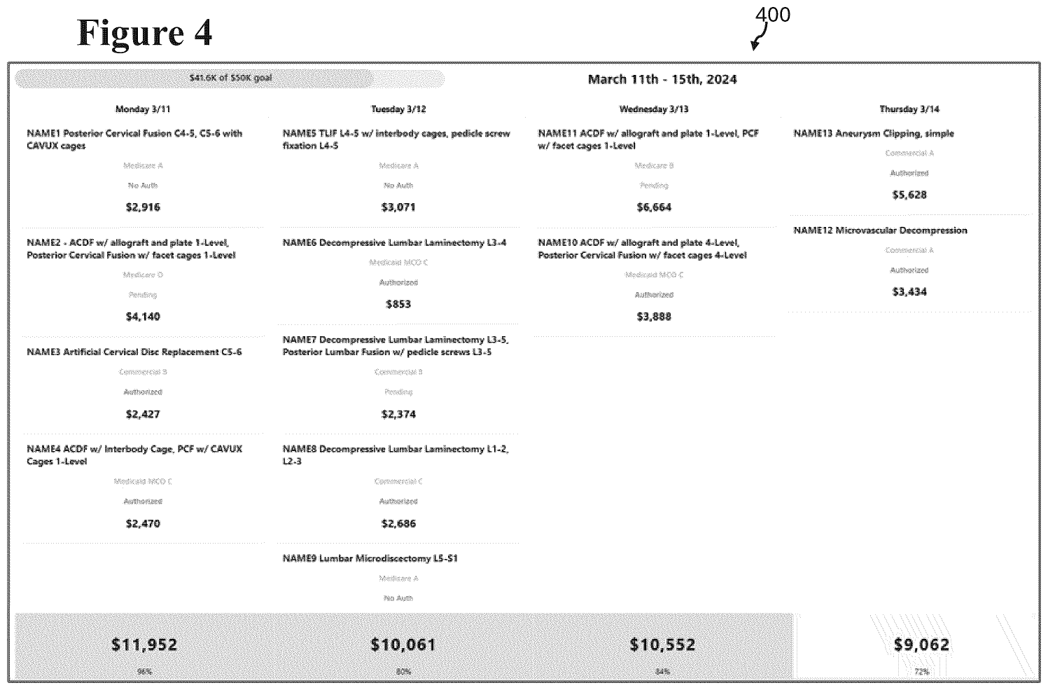

FIG. 4 illustrates a representative report 400 wherein real-time information is provided that can inform decisions toward expected and actual earnings values. Such may include calculated numbers and estimates thereof and authorization status but may also include indicators such as color to denote its relative suitability for attaining expected and actual earnings values, for example, red, yellow, and green.

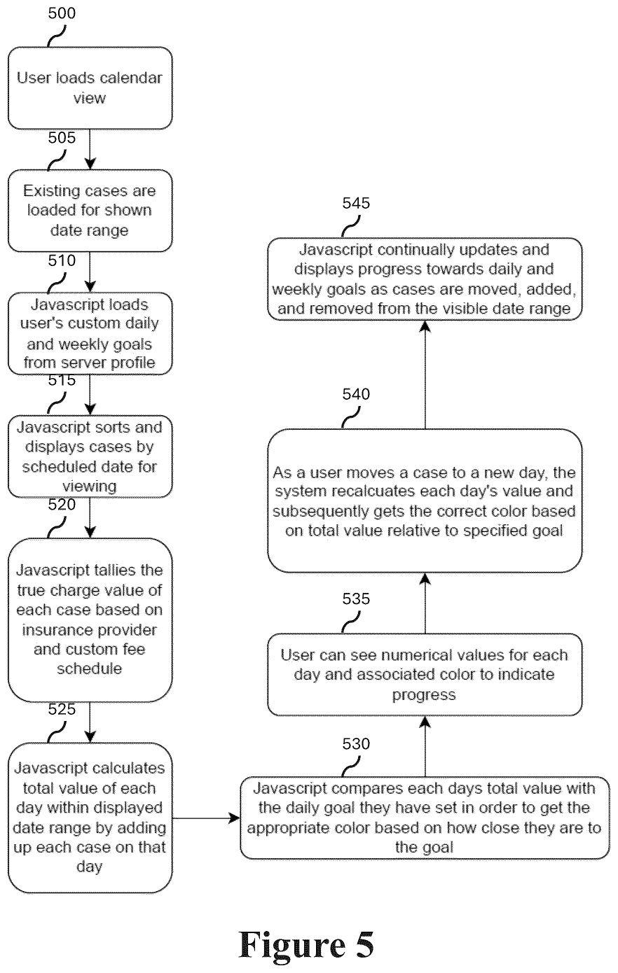

FIG. 5 illustrates an associated reporting process of the invention that includes the step of 500, user loading calendar view; the step of 505, user loading existing cases for shown date range; the step of 510, Javascript loading from server profiles for custom and weekly goals; the step of 515, Javascript sorting and displaying cases by scheduled data for viewing; the step of 520, Javascript tallying the true charge value of each case based on insurance carrier and custom fee schedule; the step of 525, Javascript calculating total value of each day within displayed date range by adding up each case on that day; the step of 530, Javascript comparing each days total value with daily earnings goal in order to get the appropriate color based on closeness to the goal; the step of 535, presenting numerical values for each day and associating color to indicate progress; the step of 540, the system recalculating each day's value and subsequently getting the correct color based on total value relative to specified goals, and the step of 545, Javascript continually updating and displaying progress toward daily and weekly goals as cases are moved, added, and removed from the visible date range, recalculating each day's value and subsequently getting the correct color based on total value relative to specified goals.

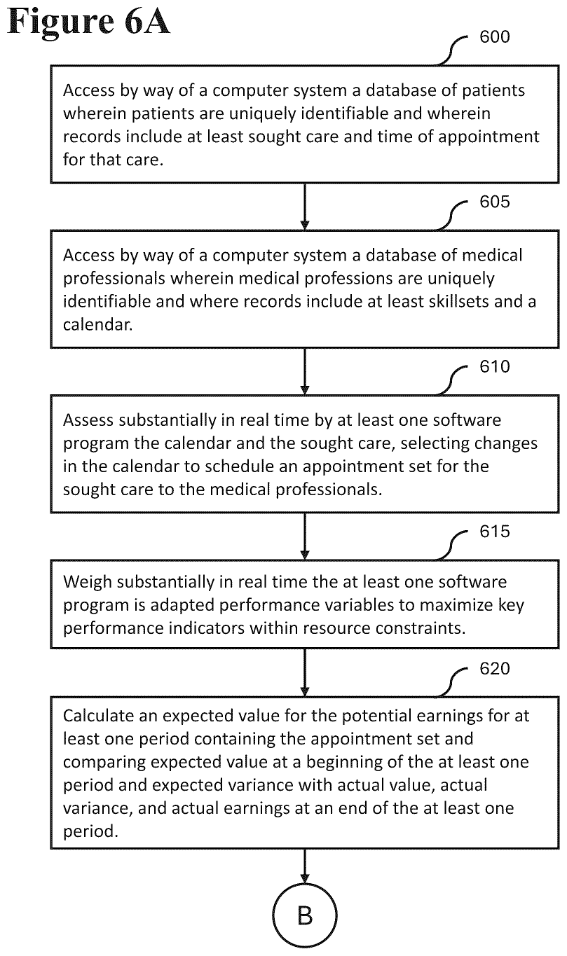

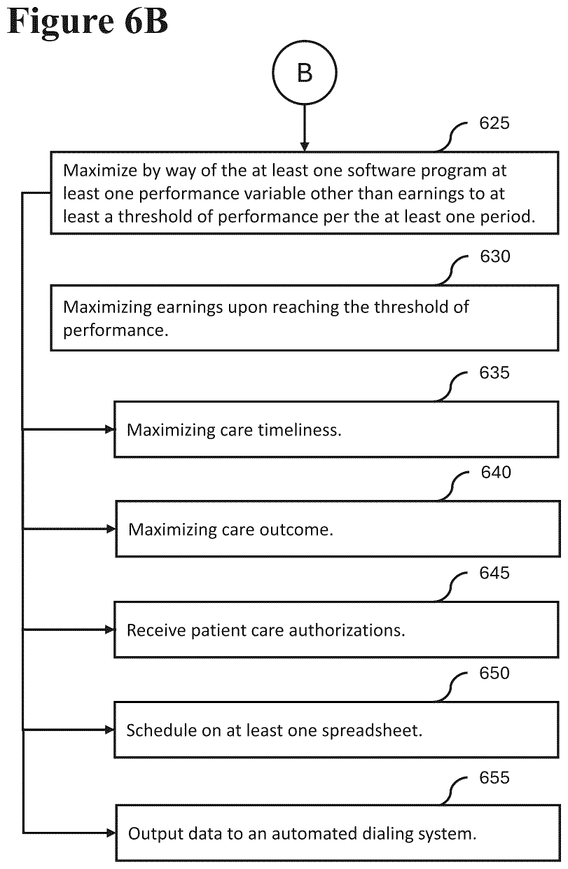

FIG. 6A-4B illustrates a representative decision support method for healthcare workflow management that includes the step of 600 accessing by way of a computer system 110 a patient database 120 wherein patients are uniquely identifiable and wherein records include at least sought procedures and sought date of appointments for those procedures. The method includes the step of 605 accessing by way of a computer system 110 a medical professional database 130 wherein medical professionals are uniquely identifiable and where records include at least skillsets and a calendar. The method includes the step of 610 assessing 163 substantially in real time by at least one software program 140 the calendar and the sought procedures, selecting changes (deciding 166) in the calendar to schedule an appointment set for the sought procedures to the medical professionals. The method includes the step of 615 weighing substantially in real time the at least one software program 140 designed to calculate performance variables to maximize key performance indicators within resource constraints. The method includes the step of 620 calculating an expected value for the potential earnings for at least one period containing the appointment set and comparing expected value at a beginning of the at least one period and expected variance with actual value and actual variance at an end of the at least one period. The method includes the step of 625 maximizing by way of the at least one software program 140 at least one performance variable other than earnings to at least a threshold of performance per the at least one period. The method includes the step of 630 maximizing earnings upon reaching the threshold of performance. Action 169 is taken from the decision wherein a procedure is performed. Orientation 162 is set for how calculations will be made for sought maximizations and minimizations within constraints.

The method may include the step of 635 maximizing care timeliness. The method may include the step of 640 maximizing care outcome score. The method may include the step of 645 receiving patient care authorizations. The method may include the step of 650 scheduling on at least one spreadsheet readable by a person. The method may include the step of 655 outputting data adapted to be used by an automated dialing system.

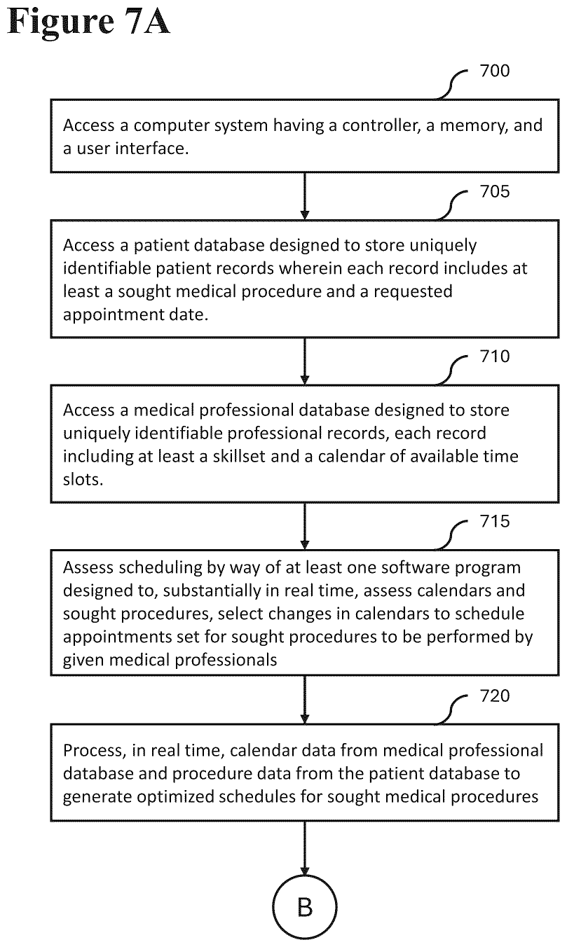

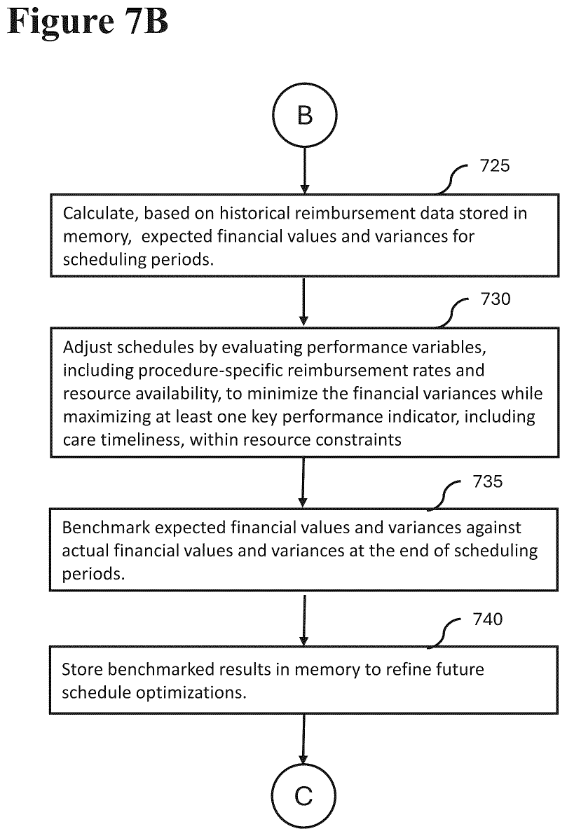

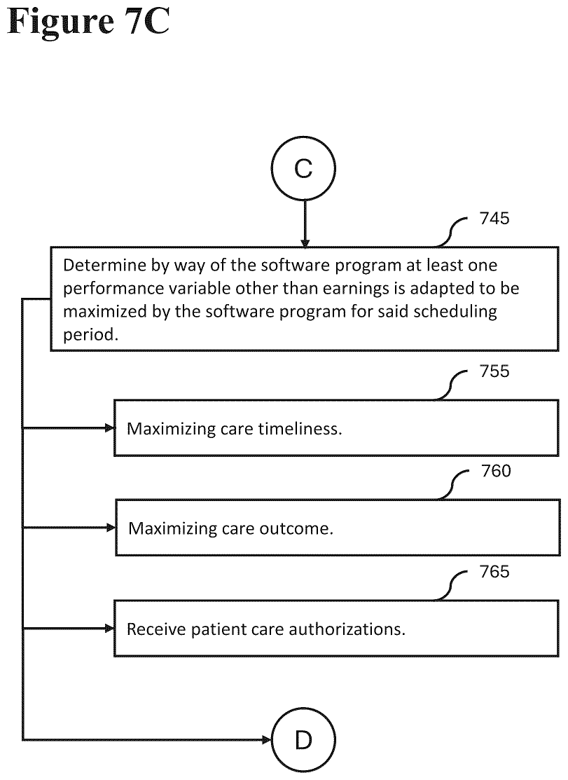

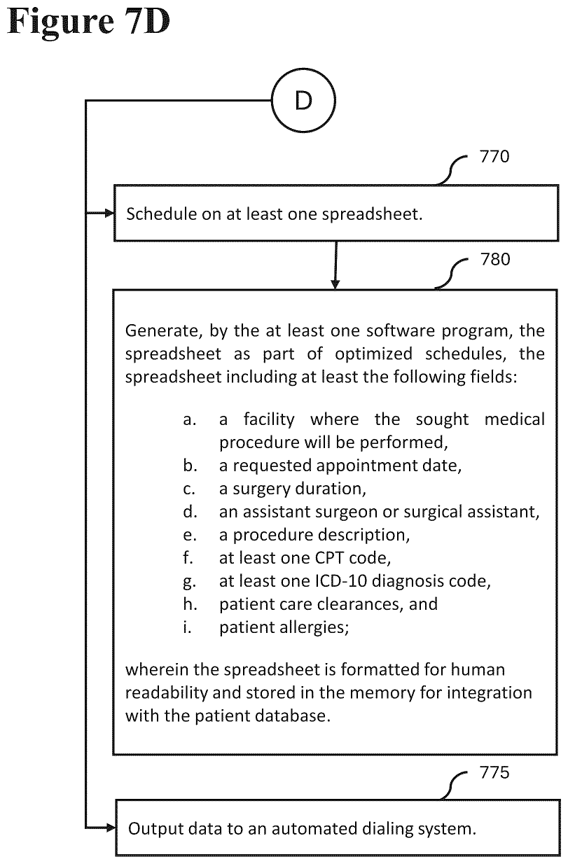

FIG. 7A-7D illustrates a second representative decision support method for healthcare workflow management, including the step of 700 accessing computer system 110 having controller 112, memory 114, and user interface 116. The method further includes the step of 705, accessing patient database 120 designed to store uniquely identifiable patient records wherein each record includes at least a sought medical procedure and a requested appointment date. The method further includes the step of 710, accessing medical professional database 130 designed to store uniquely identifiable professional records, each record including at least a skillset and a calendar of available time slots. The method further includes the step of 715, assessing scheduling by way of at least one software program 140 designed to, substantially in real time, assess calendars and sought procedures, select changes in calendars to schedule appointments set for sought procedures to be performed by given medical professionals, including;
a) 720 processing, in real time, calendar data from medical professional database 130 and procedure data from the patient database 120 to generate optimized schedules for sought medical procedures;
b) 725 calculating, based on historical reimbursement data stored in memory 114, expected financial values and variances for scheduling periods;
c) 730 adjusting schedules by evaluating performance variables, including procedure-specific reimbursement rates and resource availability, to minimize the financial variances while maximizing at least one key performance indicator, including care timeliness, within resource constraints;
d) 735 benchmarking expected financial values and variances against actual financial values and variances at the end of scheduling periods; and
c) 740 storing benchmarked results in memory 114 to refine future schedule optimizations.

The method includes the step of 745 determining by way of the software program at least one performance variable other than earnings is adapted to be maximized by the software program for said scheduling period.

The method may include the step of 755 maximizing care timeliness. The method may include the step of 760 maximizing care outcome score. The method may include the step of 765 receiving patient care authorizations. The method may include the step of 770 scheduling on at least one spreadsheet readable by a person. The method may include the step of 775 outputting data adapted to be used by an automated dialing system.

The method may further include the step of 780, generating, by the at least one software program 140, spreadsheet 150 as part of optimized schedules, spreadsheet 150 including at least the following fields as expected in the art:
a) a facility where the sought medical procedure will be performed,
b) a requested appointment date,
c) a surgery duration,
d) an assistant surgeon or surgical assistant,
e) a procedure description,
f) at least one CPT code,
g) at least one ICD-10 diagnosis code,
h) patient care clearances, and
i) patient allergies;
   wherein spreadsheet 150 is formatted for human readability and stored in the memory 114 for integration with the patient database 120.

Spreadsheet 150 is also adapted to include entries specific to given patients such as, but in no way limited to:
Type of Anesthesia: [general, local, etc.]
Position: [prone, supine, lateral, etc.]
Special Tool(s): [equipment for intraoperative diagnostic imaging,
Implant(s): [brand and model of implants used in surgery]
Implant(s) Removal: [brand and model of implants being removed during surgery]
Graft(s): [Synthetic, Autologous, or Allogeneic components used during surgery]
Intraoperative Neuromonitoring: [procedures used during surgery to assess and protect the nervous system]
Pretesting Orders: [any testing required in advance by the anesthesiologist, surgeon, and/or another physician involved in the patient's care that must be reviewed to ensure the patient can safely undergo surgery]
Day of Surgery Orders: [additional medication and anesthesia protocols to adhere to on the day of surgery or during surgery]
   Such entries represent information that may be unique to one case, types of cases, or another subset of cases.

Various related embodiments of the inventive concept are also described in the drawings, which are incorporated herein by reference in their entirety. The following patents are incorporated by reference in their entirety: Pat. Nos. US US20240046167A1

While inventive concepts have been described above in terms of specific embodiments, it is to be understood that the inventive concepts are not limited to these disclosed embodiments. Upon reading the teachings of this disclosure, many modifications and other embodiments of the inventive concepts will come to mind of those skilled in the art to which these inventive concepts pertain, and which are intended to be and are covered by both this disclosure and the appended claims. It is indeed intended that the scope of the inventive concepts should be determined by proper interpretation and construction of the appended claims and their legal equivalents, as understood by those of skill in the art relying upon the disclosure in this specification and the attached drawings.

### REPRESENTATIVE COMPONENTS

100 Decision support system
110 Computer system
112 Controller
114 Memory
116 User interface
120 Patient database
130 Medical professional database
140 Software program
150 Spreadsheet
160 Decision cycle
170 Decision Tree (Representative)
162 Orientation
163 Assess
166 Decision
169 Act
400 Report
500-545 Representative reporting process
600-655 Representative method

What is claimed is:

1. A decision support system for healthcare workflow management comprising:
a computer system having a controller, a memory, and a user interface;
a patient database adapted to store uniquely identifiable patient records wherein each record includes at least a sought medical procedure and a requested appointment date;
a medical professional database adapted to store uniquely identifiable professional records, records adapted to include at least a skillset and a calendar of available time slots;
at least one software program adapted to, substantially in real time, assess the calendar and the sought procedures, select changes in the calendar to schedule an appointment set for the sought procedures to be performed by given medical professionals;
wherein the at least one software program is executed by the controller and configured to:
a) process, in real time, the calendar data from the medical professional database and procedure data from the patient database to generate an optimized schedule for the sought medical procedures;
b) calculate, based on historical reimbursement data stored in the memory, an expected financial value and variance for a scheduling period;
c) adjust the schedule by evaluating performance variables, including procedure-specific reimbursement rates and resource availability, to minimize the financial variance while maximizing at least one key performance indicator, including care timeliness, within resource constraints;
d) benchmark the expected financial value and variance against actual financial value and variance at the end of the scheduling period; and
e) store the benchmarked results in the memory to refine future schedule optimizations;
wherein the software program is adapted to reduce computational overhead by streamlining real-time data processing, thereby improving the reliability of healthcare workflow management; and
wherein at least one performance variable other than earnings is adapted to be maximized by the software program for said scheduling period.

2. The decision support system of claim 1 wherein at least one software program is a machine learning program trained to predict optimal schedules based on historical reimbursement data.

3. The decision support system of claim 1 wherein the value maximized is timeliness of care.

4. The decision support system of claim 1 wherein the value maximized is a care outcome score.

5. The decision support system of claim 1 further including at least one software program adapted to receive patient care authorizations.

6. The decision support system of claim 1 further including at least one surgical scheduler spreadsheet.

7. The decision support system of claim 1, wherein upon at least reaching a threshold of performance for a variable other than earnings, the software program is adapted to maximize earnings.

8. A decision support system for healthcare workflow management comprising:
a computer system having a controller, a memory, and a user interface;
a patient database wherein patients are uniquely identifiable and wherein records include at least sought procedures and sought appointment dates for those procedures;
a medical professional database wherein medical professionals are uniquely identifiable and where records include at least skillsets and a calendar;
at least one software program adapted to, substantially in real time, assess the calendar and the sought procedures, select changes in the calendar to schedule an appointment set for the sought procedures to the medical professionals;
wherein the at least one software program is adapted substantially in real time to weigh performance variables to maximize key performance indicators within resource constraints;
wherein expected value and expected variance at a beginning of the at least one period are benchmarked against actual value and actual variance at an end of the at least one period;
wherein the system is adapted to maximize at least one performance variable other than earnings at least to a threshold of performance by the software program per the at least one period; and
wherein upon at least reaching the threshold of performance, the software program is adapted to maximize earnings.

9. The decision support system of claim 8 wherein at least one software program is a machine learning program trained to predict optimal schedules based on historical reimbursement data.

10. The decision support system of claim 8 wherein the value maximized is timeliness of care.

11. The decision support system of claim 8 wherein the value maximized is a care outcome score.

12. The decision support system of claim 8 further including at least one surgical scheduler spreadsheet.

13. A decision support method for healthcare workflow management comprising:
accessing a computer system having a controller, a memory, and a user interface;
accessing a patient database adapted to store uniquely identifiable patient records wherein each record includes at least a sought medical procedure and a requested appointment date;
accessing a medical professional database adapted to store uniquely identifiable professional records, each record including at least a skillset and a calendar of available time slots;
assessing scheduling by way of at least one software program adapted to, substantially in real time, assess the calendar and the sought procedures, select changes in the calendar to schedule an appointment set for the sought procedures to be performed by given medical professionals, including;
a) processing, in real time, the calendar data from the medical professional database and procedure data from the patient database to generate an optimized schedule for the sought medical procedures;
b) calculating, based on historical reimbursement data stored in the memory, an expected financial value and variance for a scheduling period;
c) adjusting the schedule by evaluating performance variables, including procedure-specific reimbursement rates and resource availability, to minimize the financial variance while maximizing at least one key performance indicator, including care timeliness, within resource constraints;
d) benchmarking the expected financial value and variance against actual financial value and variance at the end of the scheduling period; and
e) storing the benchmarked results in the memory to refine future schedule optimizations;
determining by way of the software program at least one performance variable other than earnings is adapted to be maximized by the software program for said scheduling period.

14. The decision support method of claim 13 wherein the at least one software program is a machine learning program.

15. The decision support method of claim 13 further including maximizing care timeliness.

16. The decision support method of claim 13 further including maximizing care outcome score.

17. The decision support method of claim 13 further including receiving patient care authorizations.

18. The decision support method of claim 13 further including outputting data adapted to be used by an automated dialing system.

19. The decision support method of claim 13 further including scheduling on at least one spreadsheet readable by a person.

20. The decision support method of claim 19, further comprising generating, by the at least one software program, the spreadsheet as part of the optimized schedule, the spreadsheet including at least the following fields:
a) a facility where the sought medical procedure will be performed,
b) a requested appointment date,
c) a surgery duration,
d) an assistant surgeon or surgical assistant,
e) a procedure description,
f) at least one CPT code,
g) at least one ICD-10 diagnosis code,
h) patient care clearances, and
i) patient allergies;
wherein the surgical order form is formatted for human readability and stored in the memory for integration with the patient database.
